(19) United States
(12) Patent Application Publication
(10) Pub. No.: US 2008/0033046 A1
(43) Pub. Date: Feb. 7, 2008

(54) Treatment of myocardial infarction with 11HSD1 inhibitors

(75) Inventors: Brian R. Walker, Edinburgh (GB); Gary R. Small, Edinburgh (GB); Patrick W.F. Hadoke, Edinburgh (GB)

Correspondence Address: EDWARDS ANGELL PALMER & DODGE LLP, P.O. BOX 55874, BOSTON, MA, 02205, US

(73) Assignee: University of Edinburgh, Edinburgh (GB)

(21) Appl. No.: 11/701,898

(22) Filed: Feb. 1, 2007

Related U.S. Application Data
(63) Continuation of application No. PCT/GB05/03073, filed on Aug. 4, 2005.

(30) Foreign Application Priority Data
Aug. 24, 2004 (GB) 0418877.7

(51) Int. Cl. A61K 31/19; A61K 38/43; A61P 43/00

(52) U.S. Cl. 514/569; 424/94.1

(57) ABSTRACT

The present invention relates to a method for the treatment of myocardial infarction. In particular the invention relates to the treatment of myocardial infarction using inhibitors of a particular enzyme involved in glucocorticoid metabolism.

The present invention relates to a method for the treatment of myocardial infarction. In particular the invention relates to the treatment of myocardial infarction using inhibitors of a particular enzyme involved in glucocorticoid metabolism.

### BACKGROUND

Atheromatous plaques within coronary arteries will narrow the vessel lumen, thus reducing blood flow and causing myocardial ischaemia and angina pectoris. Thrombosis on the surface of atheromatous plaques causes coronary artery occlusion and myocardial infarction, which is a major global cause of morbidity and mortality. If patients survive the first 48 h after infarction without fatal dysrrhythmia then their morbidity is largely determined by the extent of loss of contractile myocardium and subsequent remodelling of the affected ventricle. Infarcted myocardium is replaced by non-contractile scar tissue; resulting loss of cardiac function can be measured by reduced left ventricular ejection infraction (LVEF), which is predictive of subsequent prevalence of heart failure and death. A key therapeutic need is to limit the loss of left ventricular function post myocardial infarction and thus attenuate adverse remodelling.

Interventions which rapidly restore perfusion of the occluded coronary artery can reduce infarct size; these include percutaneous coronary revascularisation, and pharmacological therapy with thrombolytic and antiplatelet drugs. Infarct size is also determined by the extent of collateral blood supply, perfusing the area with blood from other coronary arteries; this is especially important when reperfusion of the occluded artery is not achieved. An important treatment strategy is to enhance collateral blood supply, to maintain perfusion around the periphery of the infarcted myocardium.

Current therapies to enhance collateral blood supply in the heart have focused on manipulating the signals which initiate angiogenesis, ie the formation of new blood vessels from existing ones. This has been undertaken in treatment of chronic ischaemia (for reviews see (1), (2)), but has not been performed in the setting of acute myocardial infarction. The influence, if any, of enhancing collateral perfusion during immediate remodelling post-myocardial infarction is unknown.

Glucocorticoids are steroid hormones which activate glucocorticoid receptors. They include endogenous cortisol and corticosterone and their 5α-reduced metabolites, and synthetic exogenous steroids such as dexamethasone, prednisolone, triamcinolone, beclomethasone. Endogenous glucocorticoid secretion is markedly increased during stress, including myocardial infarction. Glucocorticoids have potent anti-inflammatory and metabolic effects. They also regulate cellular differentiation and proliferation Glucocorticoids inhibit angiogenesis, as illustrated by their utility in the treatment of capillary haemangiomas in children ((3)). Because of their anti-inflammatory effects, it has been proposed that glucocorticoids will reduce the risks of thrombosis in coronary arteries, eg following intraluminal injury (4). Thus, administration of excess glucocorticoids has been considered to be potentially beneficial in determining the outcome of myocardial infarction.

The intracellular access of glucocorticoids to glucocorticoid receptors is controlled within the target cell by enzymes, including 11β-hydroxysteroid dehydrogenases (11HSDs) and 5α-reductases. 11HSD type 1 (11HSD1) regenerates active glucocorticoids (cortisol and corticosterone) from their inactive 11-keto-metabolites (cortisone and 11-dehydrocorticosterone, respectively). 11HSD1 is expressed in several different tissues. Its manipulation in liver, adipose tissue, macrophages and central nervous system for use in metabolic, neurodegenerative and inflammatory diseases is the subject of our patent specifications 5α-Reductase type 1 generates 5α-reduced glucocorticoid metabolites which activate glucocorticoid receptors. Its manipulation in metabolic disease and its influence on angiogenesis is the subject of patent specification.

11HSD1 is expressed in the myocardium ((5)). It has been proposed that 11HSDs in the heart prevent activation of mineralocorticoid receptors and hence inhibition of 11HSDs exacerbates glucocorticoid-induced myocardial fibrosis ((6)). 11HSD1 is 11HSD1 is expressed in the myocardium ((5)). It has been proposed that 11HSDs in the heart prevent activation of mineralocorticoid receptors and hence inhibition of 11HSDs exacerbates glucocorticoid-induced myocardial fibrosis ((6)). 11HSD1 is also expressed in vascular smooth muscle cells, where it has reductase activity (converting 11-keto metabolites to active glucocorticoids) but has apparently no effect on vascular tone ((7), (8)). However, recent data (11) show that it amplifies the anti-angiogenic effects of glucocorticoids. Increased 11HSD1 in vascular smooth-muscle cells in response to inflammatory cytokines has been suggested (9)) to provide beneficial amplification of local glucocorticoid concentrations eg during vascular injury. Conversely, loss or inhibition of 11HSD1 in vascular smooth muscle might therefore have adverse effects. 11HSD1 is also expressed in macrophages (PCT/GB02/00255) where loss of 11HSD1 has been associated with failure to clear neutrophils during sterile peritonitis; hence loss of 11HSD1 in macrophages is predicted to have an adverse effect in circumstances of acute inflammation. Thus the prior art suggests that the inhibition of 11HSD1 would be therapeutically deleterious in the treatment of conditions such as myocardial infarction.

### SUMMARY OF THE INVENTION

The present inventors have surprisingly shown that loss of 11HSD1 (associated with lower intracellular glucocorticoid concentrations) has beneficial rather than adverse effects in the treatment of myocardial infarction. Specifically, the inventors have shown that induction of myocardial infarction by coronary artery ligation in mice with 11HSD1 deficiency results in greater collateral angiogenesis and improved LVEF than in wild type control mice with intact 11HSD1.

Thus in a first aspect the present invention provides a method for the treatment of myocardial infarction in a patient which method comprises administering to a patient in need of such treatment an inhibitor of 11HSD1.

In a further aspect the invention provides the use of an inhibitor of 11HSD1 in the preparation of a medicament for the treatment of myocardial infarction in a patient.

In a further aspect the in invention provides a method for the treatment of myocardial infarction in a patient which method comprises the step of administering to a patient in need of such treatment a therapeutically effective amount of an 11HSD1 inhibitor.

In a further aspect the invention provides the use of an 11HSD1 inhibitor in the preparation of a medicament for increasing the left ventricular ejection fraction following myocardial infarction.

In yet a further aspect the invention provides the use of an inhibitor of 11HSD1 in the preparation of a medicament for enhancing myocardial remodelling post myocardial infarction in a patient.

In a further aspect still the invention provides the use of an inhibitor of 11HSD1 in the preparation of a medicament for enhancing myocardial angiogenesis following myocardial infarction in a patient.

Inhibitors of 11HSD1 are widely known in the art and include those listed by Monder and White, and those listed in the patent literature including but not limited those disclosed in the following patents and applications: WO 2004/033427, WO 2004/041264, WO 2004/011410 in the name of Astrazeneca; WO 2003/104207, WO 03/104208, WO 03/065983, WO2004058741 and US 2004/0048912 in the name of Merck, WO 2004/037254, WO02072084 in the name of Sterix and WO01990090, WO0190091, WO0190092, WO0190093, WO0190094 in the name of Biovitrum and WO 2004/056745 in the name of Janssen.

According to the above aspects of the invention, preferably the 11HSD1 inhibitor is used in conjunction with one or more agents used in the routine treatment of mycardial infarction in the preparation of a medicament for use according to the present invention.

In yet a further aspect the present invention provides a composition comprising, preferably consisting of, an inhibitor of 11HSD1 and one or more agents used in the routine treatment of myocardial infarction and a pharmaceutically acceptable carrier, diluent and/or exipient.

According to the above aspect of the invention preferably the one or more agents used routinely in the treatment of myocardial infarction is one or more of those agents in the group consisting of: thrombolytic agents; Glycoprotein IIb-IIIa platelet inhibitors, Calcium channel blockers, Antiarrythmics: Amiodarone and Lidocaine, Unfractionated heparin (UFH) or low-molecular-weight heparin (LMWH), Nitrates, Beta-blockers, Angiotensin receptor blockers, and angiotensin converting enzyme inhibitors.

According to the above aspect of the invention, advantageously the one or more 11HSD1 inhibitors are any of those described herein. Most preferably, the 11HSD1 inhibitor is carboxenolone.

In a further aspect still the present invention provides a method for the treatment of myocardial infarction in a patient which method comprises the step of administering to a patient in need of such treatment, either sequentially or simultaneously, an inhibitor of 11HSD1 in conjunction with one or more agents selected from the group consisting of the following: thrombolytic agents; Glycoprotein IIb-IIIa platelet inhibitors, Calcium channel blockers, Antiarrythmics: Amiodarone and Lidocaine, Unfractionated heparin (UFH) or low-molecular-weight heparin (LMWH), Nitrates, Beta-blockers, Angiotensin receptor antagonists and Angiotensin Converting Enzyme inhibitors.

In yet a further aspect the invention provides the use of an inhibitor of 11HSD1 in conjunction with one or more agents selected from the group consisting of the following: thrombolytic agents; Glycoprotein IIb-IIIa platelet inhibitors, Calcium channel blockers, Antiarrythmics: Amiodarone and Lidocaine, Unfractionated heparin (UFH) or low-molecular-weight heparin (LMWH), Nitrates, Beta-blockers, Angiotensin receptor antagonists and Angiotensin Converting Enzyme inhibitors in the preparation of a medicament for the treatment of mycocardial infarction in a patient.

According to the above aspects of the invention preferably the one or more agents is an anti-thromolytic agent. Most preferably the thrombolytic agent is TPA or streptokinase.

### DEFINITIONS

11HSD1: It has been shown that two iso-enzymes of 11HSD exist. Both are members of the short chain alcohol dehydrogenase (SCAD) superfamily which have been widely conserved throughout evolution. 11HSD type 2 acts as a dehydrogenase to convert the secondary alcohol group at the C-11 position of cortisol to a secondary ketone, so producing the less active metabolite cortisone. 11HSD type 1 is thought to act mainly in vivo as a reductase converting less active cortisone to more active cortisol. In vivo homozygous mice with a disrupted type 1 gene are unable to convert cortisone to cortisol, giving further evidence for the reductive activity of the enzyme. 11HSD type 1 is expressed in many key glucocorticoid regulated tissues like the liver, pituitary, gonad, brain, adipose and adrenals, however, the function of the enzyme in many of these tissues is poorly understood. Accordingly as herein defined the ‘functional activity’ of 11HSD1 refers to the activity of 11HSD1 in catalysing the reduction of cortisone to cortisol.

According to the present invention the term ‘myocardial infarction’ means heart attack, or coronary thrombus. Myocardial Infarction means the death of a muscle, tissue or organ as a result of a blockage of the blood supply to it. The heart muscle needs oxygen to survive. The coronary arteries deliver oxygenated blood to the heart muscle. When one or more of the arteries supplying the heart blocks, the oxygen supply to the myocardium stops, and the part of the heart supplied by that particular artery dies. This is a myocardial infarction.

According to the present invention the term ‘remodelling’ means the structural changes in the ventricular wall which follow myocardial infarction and includes, but is not restricted to, thinning of the ventricular wall, increase in internal ventricular diameter, reduced fractional shortening of myocardium during contraction, and reduced ejection fraction.

As herein defined the term ‘inhibitor of 11HSD1’ refers to an agent or method/technique which results in the significant inhibition of the functional activity of 11HSD1 when compared with a suitable control. The term ‘significant’ inhibition of the functional activity of 11HSD1 means greater than 20%, inhibition of the functional activity of 11HSD1 when compared with a suitable control Advantageously, an ‘inhibitor’ of 11HSD1 inhibits the functional activity of 11HSD1 by greater than 30%, 40%, 50%, 60%, 70%, 80%, 90%, 95%, 96%, 97%, 98%, 99% when compared with a suitable control. Most advantageously, an ‘inhibitor’ of 11HSD1 inhibits the functional activity of 11HSD1 by 100% when compared with a suitable control. Suitable controls for comparing the functional activity of 11HSD1 in the presence and absence of an inhibitor will be familiar to those skilled in the art. As herein defined the ‘functional activity’ of 11HSD1 refers to the activity of 11HSD1 in catalysing the reduction of cortisone to cortisol.

Inhibitors of 11HSD1 according to the present invention are advantageously agents. Agents which inhibit the functional activity of 11HSD1 may be naturally occurring or synthetic. Such agents include but are not limited to any one or more of those groups of agents consisting of the following: antibodies as herein defined; small synthetic molecules, large synthetic molecules; peptides; anti-sense nucleic acid and siRNA. Those skilled in the art will appreciate that this list is not intended to be exhaustive.

### BRIEF DESCRIPTION OF THE FIGURES AND TABLES

FIG. 1

Angiogenesis in aortic rings in vitro

(a) Light microscopy of new vessels sprouting from aortic rings

(i) Aortic ring incubated for 7 days without glucocorticoid. (ii) Aortic ring incubated for 7 days in the presence of glucocorticoid (white scale bar is 0.2 mm). Thick white arrows indicate the aortic ring; thin white arrows indicate new vessels.

(b) Uptake of LDL shown by fluorescence microscopy.

(i) This ring was incubated for 7 days without steroid. Thick white arrows indicate the aortic ring; thin white arrows indicate uptake of fluorescent labelled low density lipoprotein in endothelial cells in new vessels; black arrows indicate uptake in endothelial cells of the aortic ring (white scale bar is 0.2 mm).

(ii) High power view of new vessels; thick white arrows indicate the aortic ring and thin white arrows indicate uptake of fluorescent labelled low density lipoprotein in endothelial cells (white scale bar is 0.02 mm).

(c) Time course and effect of corticosterone on angiogenesis. Results from vessels incubated without steroids are in open symbols, and from vessels incubated with corticosterone (600 nM) in closed symbols. Results are mean ±SEM for n=4 per group. Comparison was by repeated measures ANOVA; p<0.02.

(d) Effects of corticosterone and 11dehydrocorticosterone. Vessels were counted after 7-day incubation with steroids. Results are mean SEM. #p<0.01 versus vehicle by 2-way ANOVA and LSD post hoc test.

(e) Effects of the mineralocorticoid receptor antagonist spironolactone. Aortic rings from C57B16 mice were incubated with (filled columns) and without (open columns) spironolactone (10⁻⁶ M) and glucocorticoids (600 nM). Results are mean ±SEM for n=6 experiments. # p<0.02 versus corresponding vehicle. Spironolactone alone had no effect.

(f) Effects of the glucocorticoid receptor antagonist RU38486. Aortic rings from C57B16 mice were incubated with (filled columns) and without (open columns) RU38486 (10⁻⁶ M) and glucocorticoids (600 nM). Results are mean ±SEM for n=4-6 experiments. # p<0.01 versus corresponding vehicle. ***p<0.001 for the effect of RU38486 in the presence of glucocorticoid. RU38486 alone had no effect.

(g) Effects of the 11HSD inhibitor carbenoxolone. Aortic rings from C57B16 mice were incubated with (filled columns) and without (open columns) carbenoxolone (10⁻⁶ M) and glucocorticoids (600 nM). Results are mean ±SEM for n=5 experiments. # p<0.01 versus corresponding vehicle. * p<0.04 for the effect of carbenoxolone in the presence of 11-dehydrocorticosterone. Carbenoxolone had no effect in the presence of corticosterone or vehicle alone.

(h) Effects of corticosterone and 11-dehydrocorticosterone on angiogenesis in vessels from 11HSD1 −/− mice. Aortic rings from C57B16 wild type (open columns) or 11bHSD1 −/− (filled columns) mice were incubated with and without glucocorticoids (600 nM); Results are mean ±SEM for n=7 experiments. # p<0.01 versus corresponding vehicle. **p<0.01 for differences in angiogenesis between wild type and 11bHSD1 −/− mice. Angiogenesis was not different between strains in the presence of vehicle or corticosterone but was inhibited by 11-dehydrocorticosterone in wild type but not 11bHSD1 −/− mice.

FIG. 2

Angiogenesis in subcutaneous implanted sponges

(a) Light microscopy (×10) of haematoxylin and eosin stained sponge 8 m sections from wild type mice: (i) vehicle and (ii) cortisol treated sponge. Both sponges were covered with a fibroblast rich fibrous coat and were infiltrated with inflammatory neutrophils and lymphocytes. Placebo treated sponges alone were also infiltrated with an organised matrix and an abundance of blood vessels (dark arrows).

(b) Sponges from C57B16 wild type (n=6) with (filled columns) or without RU38486 (open columns). Results are mean ±SEM. # p<0.01 versus vehicle. New vessel formation was greater in RU38486 impregnated sponges versus their contra lateral controls.

(c) Sponges from C57B16 wild type (open columns n=12) or 11bHSD1 −/− (filled columns n=6) mice with and without glucocorticoids. Results are mean ±SEM. # p<0.001 versus corresponding vehicle. *** p<0.001 for differences between wild type and 11bHSD1 −/−. Placebo impregnated sponges exhibited an increased angiogenic response in 11bHSD1 −/− compared to wild type mice. Cortisol inhibited angiogenesis in both strains but cortisone inhibited angiogenesis only in wild type mice.

FIG. 3

Effect of chronic coronary artery ligation on angiogenesis in mouse myocardium

(a) Light microscopy of haematoxylin and eosin stained hearts; 8 m sections from (i) wild type and (ii) 11bHSD1 −/− mice 7 days post ligation (lv, left ventricle; wd, width). Infarcted left ventricular wall is thinned in comparison to sham-operated animals but relatively preserved in 11bHSD1 −/− mice. Light microscopy (×50) of anti-von Willebrand factor immunostaining with fast red chromogen substrate in day 7 sham (iii) and infarcted (iv) hearts. Scattered medium and large vessels were detected in sham hearts in contrast many more vessels were observed in the healing myocardium post-infarction. Black arrows indicate vessels, lv is left ventricle, es is endocardial surface

(b) Vascularity of myocardium of wild type mouse hearts following ligation (filled columns n=2-7) or sham surgery (open columns n=1-4). Sham operated animals show a constant vascularity in contrast to CCL animals in whom vessel counts increase with time achieving a maximum at day 7.

(c) Hearts from ligated (filled columns) or sham operated wild type (n=2 sham, n=7 ligations) and 11bHSD1 −/− (n=3 sham, n=5 ligations) mice. Results are mean ±SEM. # p<0.001 versus corresponding sham. ***p<0.001 for differences between wild type and 11bHSD1 −/−. An increased angiogenic response occurred after coronary artery ligation 11bHSD1 −/− compared to wild type mice.

FIG. 4 shows angiogenesis 7 days post myocardial infarction in wild type (n=7) and 11HSD1 knock out mice (n=5). Specifically it shows the numbers of myocardial vessels identified in the two test groups following sham surgery and following coronary artery ligation. Details are provided in Example 4.

FIG. 5 shows left ventricular function 7 days post chronic coronary ligation in wild type (n=5) and 11HSD1 knock out mice (n=5). Specifically it shows the left ventricular ejection fraction in the two groups following sham surgery and following coronary artery ligation. * indicates significant difference versus sham surgery. Details are given in Example 5.

### TABLE 1

Cortisol concentration in sponge homogenates from wild type and 11HSD1 −/− homozygous null mice. Results are mean ±SEM for n=3-6 experiments. # p<0.01 versus contralateral placebo. **p0.01 for differences between wild type and 11bHSD1

### TABLE 2

Echocardiographic measurement of left ventricular parameter from wild type and 11 bHSD1 −/− null mice 7 days after coronary artery ligation or sham surgery. Results are mean ±SEM for n=3-5 experiments. #p<0.05 for differences between infarct and relevant sham control; **p<0.01 and *p<0.05 for differences between wild type and 11bHSD1 −/−. 11bHSD-1 −/− mice showed less severe impairment in left ventricular function following coronary artery ligation. LV left ventricle, EDD end diastolic diameter, ESD end systolic diameter, FS fractional shortening ([LVEDD−LVESD]/LVEDD×100), EF ejection fraction ([LVEDarea−LVES area]/LVED area×100).

### TABLE 3

Steroid inhibitors of 11HSD1: Reproduced from Monder and White.

### DETAILED DESCRIPTION OF THE INVENTION

The Role of Glococorticoids.

Glucocorticoids are synthesised in the adrenal cortex from cholesterol. The principle glucocorticoid in the human body is cortisol, this hormone is synthesised and secreted in response to the adrenocortictrophic hormone (ACTH) from the pituitary gland in a circadian, episodic manner, but the secretion of this hormone can also be stimulated by stress, exercise and infection. Cortisol circulates mainly bound to transcortin (cortisol binding protein) or albumin and only a small fraction is free (5-10%) for biological processes.

Cortisol has a wide range of physiological effects, including regulation of carbohydrate protein and lipid metabolism, regulation of normal growth and development, influence on cognitive function, resistance to stress and mineralocorticoid activity. Cortisol works in the opposite direction compared to insulin meaning a stimulation of hepatic gluconeogenesis, inhibition of peripheral glucose uptake and increased blood glucose concentration. Glucocorticoids are also essential in the regulation of the immune response. When circulating at higher concentrations glucocorticoids are immunosuppressive and are used pharmacologically as anti-inflammatory agents.

Glucocorticoids like other steroid hormones are lipophilic penetrate the cell membrane freely. Cortisol binds, primarily, to the intracellular glucocorticoid receptor (GR) that then acts as a transcription factor to induce the expression of glucocorticoid responsive genes, and as a result of that protein synthesis.

Ischaemic Heart Disease.

Ischaemic heart disease is caused by an imbalance between the myocardial blood flow and the metabolic demand of the myocardium. Reduction in coronary blood flow is related to progressive atherosclerosis with increasing occlusion of coronary arteries. Blood flow can be further decreased by superimposed events such as vasospasm, thrombosis, or circulatory changes leading to hypoperfusion.

Coronary artery perfusion-depends upon the pressure differential between the ostia (aortic diastolic pressure) and coronary sinus (right atrial pressure). Coronary blood flow is reduced during systole because of Venturi effects at the coronary orifices and compression of intramuscular arteries during ventricular contraction.

Factors reducing coronary blood flow include:
1. Decreased aortic diastolic pressure
2. Increased intraventricular pressure and myocardial contraction
3. Coronary artery stenosis, which can be further subdivided into the following etiologies:
   Fixed coronary stenosis
   Acute plaque change (rupture, hemorrhage)
   Coronary artery thrombosis
   Vasoconstriction
4. Aortic valve stenosis and regurgitation
5. Increased right atrial pressure
40 micron collateral vessels are present in all hearts with pressure gradients permitting flow, despite occlusion of major vessels. In general, the cross-sectional area of the coronary artery lumen must be reduced by more than 75% to significantly affect perfusion. Coronary atherosclerosis is diffuse (involving more than one major arterial branch) but is often segmental, and typically involves the proximal 2 cm of arteries (epicardial).
   Myocardial Infarction.

Myocardial infarction means heart attack, or coronary thrombus. Myocardial infarction means the death of a muscle, tissue or organ as a result of a blockage of the blood supply to it.

The heart muscle needs oxygen to survive. The coronary arteries deliver oxygenated blood to the heart muscle. When one or more of the arteries supplying the heart blocks, the oxygen supply to the myocardium stops, and the part of the heart supplied by that particular artery dies. This is a myocardial infarction.

The pathogenesis can include:
Occlusive intracoronary thrombus—a thrombus overlying an ulcerated or fissured stenotic plaque causes 90% of transmural acute myocardial infarctions.
Vasospasm—with or without coronary atherosclerosis and possible association with platelet aggregation.
Emboli—from left sided mural thrombosis, vegetative enodocarditis, or paradoxical emboli from the right side of heart through a patent foramen ovale.

The gross morphologic appearance of a myocardial infarction can vary. Patterns include:
Transmural infarct—involving the entire thickness of the left ventricular wall from endocardium to epicardium, usually the anterior free wall and posterior free wall and septum with extension into the RV wall in 15-30%. Isolated infarcts of RV and right atrium are extremely rare.
Subenidocardial infarct—multifocal areas of necrosis confined to the inner ⅓-½ of the left ventricular wall. These do-not show the same evolution of changes seen in a transmural MI.

Gross morphologic changes evolve over time as follows:

| Time from | | |
|---|---|---|
| Onset | | Gross Morphologic Finding |
| 18-24 | Hours | Pallor of myocardium |
| 24-72 | Hours | Pallor with some hyperemia |
| 3-7 | Days | Hyperemic border with central yellowing |
| 10-21 | Days | Maximally yellow and soft with vascular margins |
| 7 | weeks | White fibrosis |

Microscopic morphologic changes evolve over time as follows:

| Time from | | |
|---|---|---|
| Onset | | Microscopic Morphologic Finding |
| 1-3 | Hours | Wavy myocardial fibers |
| 2-3 | Hours | Staining defect with tetrazolium or basic fuchsin dye |
| 4-12 | Hours | Coagulation necrosis with loss of cross striations, |
| | | contraction bands, edema, hemorrhage, and early |
| | | neutrophilic infiltrate |
| 18-24 | Hours | Continuing coagulation necrosis, pyknosis of nuclei, |
| | | and marginal contraction |
| | | bands |
| 24-72 | Hours | Total loss of nuclei and striations along with heavy |
| | | neutrophilic infiltrate |
| 3-7 | Days | Macrophage and mononuclear infiltration begin, |
| | | fibrovascular response begins |
| 4-21 | Days | Fibrovascular response with prominent granulation |
| | | tissue |
| 7 | Weeks | Fibrosis |

The above gross and microscopic changes over time can vary. In general, a larger infarct will evolve through these changes more slowly than a small infarct. Clinical complications of myocardial infarction will depend upon the size and location of the infarction, as well as pre-existing myocardial damage. Complications can include:
Arrhythmias and conduction defects, with possible “sudden death”
Extension of infarction, or re-infarction
Congestive heart failure (pulmonary edema)
Cardiogenic shock
Pericarditis
Mural thrombosis, with possible embolization
Myocardial wall rupture, with possible tamponade
Papillary muscle rupture, with possible valvular insufficiency
Ventricular aneurysm formation

Sudden death is defined as death occurring within an hour of onset of symptoms. Such an occurrence often complicates ischemic heart disease. Such patients tend to have severe coronary atherosclerosis (>75% luminal narrowing). Often, a complication such as coronary thrombosis or plaque hemorrhage or rupture has occurred. The mechanism of death is usually an arrhythmia.

According to the present invention the term ‘remodelling’ means the structural changes in the ventricular wall which follow myocardial infarction and includes, but is not restricted to, thinning of the ventricular wall, increase in internal ventricular diameter, reduced fractional shortening of myocardium during contraction, and reduced ejection fraction.

Routine Treatment of Myocardial Infarction.

Several drugs are presently used for the treatment of myocardial infarction. The following represents a summary of recommended treatment regimes at the filing date of the application.

Acute Adjunctive Medications

Antiplatelets, (75 mg, 150 mg, 300 mg, are UK doses) 81, 160, 325 mg aspirin (ASA) or 300 mg clopidogrel (Plavix®) and (75 mg) 81 mg ASA, given as soon as possible. Acute ASA should be withheld only from patients with true anaphylactic allergy.
Many institutions prefer to avoid early clopidogrel in primary PCI and medical therapy of patient. With AMI, as well as non-ST-segment elevation myocardial infarction (STEMI)/unstable angina acute coronary syndrome (ACS) patients, until it is clear there is no indication for surgical revascularization. Up-front glycoprotein IIb-IIIa inhibitor use is clinically indicated in this setting and avoids clopidogrel's prolonged platelet inhibition in patients who may demonstrate a surgical indication. The 2002 update for the American College of Cardiology (ACC)/American Heart Association (AHA) guideline on unstable angina (UA)/non-ST segment elevation myocardial infarction (Non-STEMI) directs treatment with clopidogrel should be started on admission unless a surgical indication is suspect.
Beta-blockers, such as metoprolol (Lopressor®), 5 mg IV every 5 minutes for three doses, followed by 25 to 50 mg orally every 6 hours *(Unless emergent need to reduce systolic blood pressure dosing tends to be orally and in the said amounts but 12 hourly not 6 hourly)* for 48 hours, then 50 to 100 mg orally twice a day. Relative contraindications include systolic blood pressure <100 mm Hg, heart rate <60 beats/min, reactive airway disease, and heart block greater than first degree.
Nitrates. A mortality benefit has not been clearly demonstrated for acute nitrate therapy, but the sublingual and intravenous forms may help reduce infarction symptoms and be useful in acute treatment of CHF and hypertension. The dose should be adjusted to relieve symptoms and maintain systolic blood pressure at >90 mm Hg. Hypotension and/or bradycardia may occur more often with nitrate use in patients with inferior myocardial infarction.
Unfractionated heparin (UFH) or low-molecular-weight heparin (LMWH). UFH or LMWH is indicated in all STEMI and non-STEMI infarcts except when streptokinase is used. Use of heparin is required with use of tissue-type plasminogen activator (tPA) reteplase (r-Pa) and tenecteplase (TNK).
Antiarrythmics: Amiodarone and Lidocaine. Amiodarone is the current drug of choice for the management of ventricular tachycardia (VT) and ventricular fibrillation (VF) according to Advanced Cardiac Life Support (ACLS) Guidelines.
Magnesium, 1 g by slow IV push, then 15 g over 24 hours.
Calcium channel blockers. They may be used in patients with contraindications to beta blockers for control of postinfarction angina or hypertension. They may compound the toxicity of beta blockers or digoxin (Lanoxicaps®, Lanoxin®) and should be used cautiously in conjunction with these agents.
Glycoprotein IIb-IIIa platelet inhibitors. Maximal medication therapies including aspirin, intravenous/oral beta-blocker, heparin and nitrates should be aggressively employed in acute coronary syndrome (ACS) patients with refractory unstable angina, usually with evidence of ischemia by ST depression or positive troponin.
Thrombolytic therapy with agents (thrombolytic agents) such as streptokinase or tissue plasminogen activator (TPA) is often used to try and lyse a recently formed thrombus. Such therapy with lysis of the thrombus can re-establish blood flow in a majority of cases. This helps to prevent significant myocardial injury, if early (less than an hour or so) in the course of events, and can at least help to reduce further damage.
Angiotensin Receptor blockers such as Losartan 50 mg orally daily and Angiotensin Converting Enzyme Inhibitors such as Ramipril 2.5-10 mg orally daily are employed especially in patients with impaired left ventricular ejection fraction after myocardial infarction.

Thus in a further aspect the present invention provides a method for the treatment of myocardial infarction in a patient which method comprises the step of administering to a patient in need of such treatment an inhibitor of 11HSD1 either sequentially or simultaneously with one or more agents selected from the group consisting of the following: thrombolytic agents; Glycoprotein IIb-IIIa platelet inhibitors, Calcium channel blockers, Antiarrythmics: Amiodarone and Lidocaine, Unfractionated heparin (UFH) or low-molecular-weight heparin (LMWH), Nitrates, Beta-blockers, Angiotensin Receptor Blockers and Angiotension Converting Enzyme inhibitors.

Advantageously, the treatment of myocardial infraction according to the method of the invention comprises the treatment of myocardial infarction using an 11HSD1 inhibitor disclosed herein either sequentially or simultaneously with any one or more of the agents described above and which are routinely used for the treatment of myocardial infarction.

In a particularly preferred embodiment of the above aspect of the invention the method comprises the use of an inhibitor of 11HSD1 either sequentially or simultaneously with one or more thrombolytic agents in the preparation of a medicament for the treatment of myocardial infarction in a patient.

Advantageously, the thrombolytic agent is TPA or streptokinase.

According to the present invention the use of one or more agent/s used to routinely treat myocardial infarction in conjunction with one or more 11HSD1 inhibitors in the preparation of a medicament for the treatment of myocardial infarction in a patient are also contemplated. Advantageously the one or more 11HSD1 inhibitors are as herein described.

11HSD1 Enzyme.

The conversion of cortisol (F) to its inactive metabolite cortisone (E) by 11HSD1 was first described in the 1950's, however it was not until later that the biological importance for this conversion was suggested. In 1983 Krozowski et al. showed that the mineralocorticoid receptor (MR) has equal binding affinities for glucocorticoids and mineralocorticoids. Because the circulating concentration of cortisol is a 100 times higher than that of aldosterone and during times of stress or high activity even more, it was not clear how the MR remained mineralocorticoid specific and was not constantly occupied by glucocorticoids. Earlier Ulick et al. had described the hypertensive condition known as “apparent mineralocorticoid excess” (AME), and observed that whilst secretion of aldosterone from the adrenals was in fact low the peripheral metabolism of cortisol was disrupted. These discoveries lead to the suggestion of a protective role for the enzymes. By converting cortisol to cortisone in mineralocorticoid dependent tissues 11HSD enzymes protects the MR from occupation by glucocorticoids and allows it to be mineralocorticoid specific. Aldosterone itself is protected from the enzyme by the presence of an aldehyde group at the C-18 position.

Congenital defects in the 11HSD enzyme results in over occupation of the MR by cortisol and hypertensive and hypokalaemic symptoms seen in AME.

Localisation of the 11HSD showed that the enzyme and its activity is highly present in the MR dependent tissues, kidney and parotid. However in tissues where the MR is not mineralocorticoid specific and is normally occupied by glucocorticoids, 11HSD is not present in these tissues, for example in the heart and hippocampus. This research also showed that inhibition of 11HSD caused a loss of the aldosterone specificity of the MR in these mineralocorticoid dependent tissues.

It has been shown that two iso-enzymes of 11HSD exist. Both are members of the short chain alcohol dehydrogenase (SCAD) superfamily which have been widely conserved throughout evolution. 11HSD type 2 acts as a dehydrogenase to convert the secondary alcohol group at the C-11 position of cortisol to a secondary ketone, so producing the less active metabolite cortisone. 11HSD type 1 is thought to act mainly in vivo as a reductase, that is in the opposite direction to type 2. 11 HSD type 1 and type 2 have only a 30% amino acid homology.

The direction of 11HSD type 1 reaction in vivo is generally accepted to be opposite to the dehydrogenation of type 2. In vivo homozygous mice with a disrupted type 1 gene are unable to convert cortisone to cortisol, giving further evidence for the reductive activity of the enzyme. 11HSD type 1 is expressed in many key glucocorticoid regulated tissues like the liver, pituitary, gonad, brain, adipose and adrenals, however, the function of the enzyme in many of these tissues is poorly understood.

The concentration of cortisone in the body is higher than that of cortisol, cortisone also binds poorly to binding globulins, making cortisone many times more biologically available. Although cortisol is secreted by the adrenal cortex, there is a growing amount of evidence that the intracellular conversion of E to F may be an important mechanism in regulating the action of glucocorticoids.

It may be that 11HSD type 1 allows certain tissues to convert cortisone to cortisol to increase local glucocorticoid activity and potentiate adaptive response and counteracting the type 2 activity that could result in a fall in active glucocorticoids. Potentiation of the stress response would be especially important in the brain and high levels of 11HSD type 1 are found around the hippocampus, further proving the role of The enzyme.

Inhibitors of 11HSD1.

As herein defined the term ‘inhibitor of 11HSD1’ refers to an agent or method/technique which results in the significant inhibition of the functional activity of 11HSD1 when compared with a suitable control. The term ‘significant’ inhibition of the functional activity of 11HSD1 means greater than 20%, inhibition of the functional activity of 11HSD1 when compared with a suitable control. Advantageously, an ‘inhibitor’ of 11HSD1 inhibits the functional activity of 11HSD1 by greater than 30%, 40%, 50%, 60%, 70%, 80%, 90%, 95%, 96%, 97%, 98%, 99% when compared with a suitable control. Most advantageously, an ‘inhibitor’ of 11HSD1 inhibits the functional activity of 11HSD1 by 100% when compared with a suitable control. Suitable controls for comparing the functional activity of 11HSD1 in the presence and absence of an inhibitor will be familiar to those skilled in the art.

As herein defined the ‘functional activity’ of 11HSD1 refers to the activity of 11HSD1 in catalysing the reduction of cortisone to cortisol.

Inhibitors of 11HSD1 according to the present invention are advantageously agents. Agents which inhibit the functional activity of 11HSD1 may be naturally occurring or synthetic. Such agents include but are not limited to any one or more of those groups of agents consisting of the following: antibodies as herein defined; small synthetic molecules, large synthetic molecules; peptides; anti-sense nucleic acid and siRNA. Those skilled in the art will appreciate that this list is not intended to be exhaustive.

Small molecule inhibitors of 11HSD1 are well known in the literature. They are many and varied in structure and characteristics. One important of 11HSD1 inhibitors are steroid inhibitors. Such inhibitors are extensively reviewed in Monder and White, 1983 which is herein incorporated by reference. The Table below (Table 1) shows some typical steroid inhibitors which may be suitable for use according to the present invention.

TABLE 3
Steroid inhibitors of 11HSD1 (Table IV, Monder and White
(1983), Vitamins and hormones vol 43, Academic Press Inc)
CARL MONDER AND PERRIN C. WHITE
STEROID INHIBITORS OF 11β-HYDROXSTEROID
DEHYDROGENASE
11β-HYDROXSTEROID DEHYDROGENASE (
(a) Oxidation (11-OH→11-oxo)
C₂₁ and C₁₉ steroids

| | |
|---|---|
| 11α,17,21-Trihydroxy•pregn-4-en- | Burton (1965) |
| 3-one (11-epicortisol) | |
| 11α,17,21-Trihydroxy•pregn-1,4- | Burton (1965) |
| diene-3-one (11-epiprednisolone) | |
| 11α-Hydroxypregn-4-en-3-one | Burton (1965); |
| (11α-hydroxyprogesterone) | Murphy and Vedady (1982) |
| 17,21.Dihydroxypregn-4-ene-3,11-dione | Bernal et al (1980); |
| (cortisone) | Murphy (1979b) |
| Cortisol 21-acetate | Bernal et al (1980) |
| Progesterone | Bernal et al (1980); |
| | Murphy and Vedady (1982) |
| 1Dehydro-16α-methyl 9α | Bernal et al. (1980) |
| fluorohydrocortisone (dexamethasone) | |
| 11β17,21-Trihydroxypregn-1,4-dien- | Bernal et al (1980); |
| 3-one (prednisolone) | Murphy and Vedady (1982) |
| 9α-Fluorocortisol | Bush et al (1968) |
| 3α,11β,17,21-Tetrahydroxy- | Deck x and DeMoor (1966) |
| 6α-pregnan-20-[ ] | |
| 3α,11β,17,20β,21_Pentahydroxy- | Deck x and DeMoor (1966) |
| 5α-pregnane (allocortol) | |
| 11β,17α,21-Trihydroxy-5α-pregnane- | Deck x and DeMoor (1966) |
| 3,20-dione (all-dehydrocortisol) | |
| 11β-Hydroxytestosterone | Monder and Lakshmi (1989a) |
| 11β-Hydroxyandrost-4-ene-3,17-dione | Deck x and DeMoor (1966); |
| | Monder and Lakshmi (1989a) |
| 3α,11β,17β-Trihdroxyandrostane | Monder and Lakshmi (1989a) |
| 3β-Hydroxyandrost-5-en-17-one | Deck x and DeMoor (1966) |
| 11β,17β-Dihydroxy-5β-androstan-3-one | Monder and Lakshmi (1989a) |
| 11β,17β-Dihydroxy-5α-androstan-3-one | Monder and Lakshmi (1989a) |

(b) Reduction (11-oxo→11-OH)
C₂₁ and C [symbol] steroids

| | |
|---|---|
| 11-Oxoprogesterone | Torday et al. (1975) |
| 3α,17,21-Trihydroxy-5β-pregnan- | Bernal et al. (1980) |
| 3-20-dione (tetrahydrocortisone) | |
| 21-Hydroxy-pregn-4-ene-3,11,20-trione | Bernal et al. (1980) |
| Androst-4-ene-3,11,20-trione | Deck x and DeMoor (1966) |
| 3β-Hydroxyandrost-5-en-17-one | Deck x and DeMoor (1966) |

(c) Do not inhibit (11β-OH→11-oxo)
C₂₁ steroids

| | |
|---|---|
| 21-Hydroxypregn-4-ene-3,20-dione | Murphy and Vedady (1982) |
| 17α,21-Dihydroxypregn-4-ene-3,11-dione | Murphy and Vedady (1982) |
| 11β-Hydroxypregn-4-ene-3,20-dione- | Murphy and Vedady (1982) |
| 21-sulfate | |
| 11β,17α-Dihydroxypregn-4-ene-3,20- | Murphy and Vedady (1982) |
| dione-21-sulfate | |
| 6α-Hydroxypregn-4-ene-3,20-dione | Murphy and Vedady (1982) |
| 12α-Hydroxypregn-4-ene-3,2-dione | Murphy and Vedady (1982) |
| 6β,11β,17α,21-Tetrahydroxypregn- | Murphy and Vedady (1982) |
| 4-ene-3,20-dione | |
| 11β21-Dihydroxy-18-oxo-pregn-4-ene- | Murphy and Vedady (1982) |
| 3,20-dione | |
| 15α-Hydroxypregn-4-en-3-one | Murphy and Vedady (1982) |
| 16α-Hydroxypregn-4-en-3-one | Murphy and Vedady (1982) |
| 3,20-Dioxo-pregn-4,16-diene | Murphy and Vedady (1982) |
| 3α,20α-Dihydroxy\hat{ }β-\hat{ } | Murphy and Vedady (1982) |
| 9α-Fluoro-11β,17α21,trihydroxy- | Murphy and Vedady (1982) |
| 16β-methylpregn-1,4-diene-3,20-Dione | |
| Tetrahydrocortisol | Bernal et al. |
| | Deck x and DeMoor (1966) |
| 3α,11β,17,20α,21-Pentahydroxy- | Deck x and DeMoor (1966) |
| 5β pregnane (α-cortol) | |
| 3α,11β,17,20β,21-Pentahydroxy- | Deck x and DeMoor (1966) |
| 5β pregnane (β-cortol) | |
| Tetrahydrocortisone | Bernal et al. (1980); |
| | Deck x and DeMoor (1966) |
| 2α-Methylcortisol | Bush et al. (1968) |

C₁₉ steroids

| | |
|---|---|
| 3β,11β-Dihydroxy-5α-androstan-17-one | Murphy and Vedady (1982) |
| 3α,11β-Dihydroxy-5α-androstan-17-one | Murphy and Vedady (1982) |
| 3β,11β-Dihydroxy-5β-androstan-17-one | Murphy and Vedady (1982) |
| 3β,11β,16α-Trihydroxyandrost- | Murphy and Vedady (1982) |
| 5-en-17-one-5β | |
| 3α,11β-Dihydroxy-5β-androstan-17-one | Monder and Lakshmi (1989a); |
| | Murphy and Vedady (1982) |
| 3β-Hydroxy-androst-5-en-17-one-3-sulfate | Murphy and Vedady (1982) |
| Testosterone | Bernal et al. (1980) |
| 5α-Dihydrotestosterone | Bernal et al. (1980) |
| 3α-Hydroxy-5β-androstan-17 | Deck x and DeMoor (1966) |
| 3α-Hydroxy-5α-androstan-17-one | Deck x and DeMoor (1966) |
| Androstenedione | Deck x and DeMoor (1966) |
| Audrost-4-ene-3,11,17-trione | Dec x and DeMoor (1966) |
| Dihydroepiandrosterone | Pepe and Albrecht (1984a) |
| 11β-Hydroxy-5β-androstane | Monder and Lakshmi (1989a) |
| 3α,11β-Dihydroxyandrosten-17-one | Monder and Lakshmi (1989a) |

C₁₈

| | |
|---|---|
| Eatradiol | Bernal et al. (1980); |
| | Abramovitz et al. (1984) |
| Eatriol | Bernal et al. (1980); |
| | Abramovitz et al. (1984) |
| Eatrone | Abramovitz et al. (1984) |

(d) Do not inhibit (11-oxo→11.OH)
C₂₁

| | |
|---|---|
| 2α-Methylcortisone | Bush et al. (1968) |
| Cortisol | Bush et al. (1968) |
| 20β-Cortol | Bush et al. (1968) |
| 20α-Cortol | Bush et al. (1968) |
| 3α,11β,17,20β-21-Pentahydroxy- | Bush et al. (1968) |
| 5α-pregnan-20-one (allocortol) | |
| Cortolone | Bush et al. (1968) |

C₁₉

| | |
|---|---|
| Androst-4-ene-3,17-dione | Bush et al. (1968) |
| 3α-Hydroxy-5α-androstan-17-one | Bush et al. (1968) |
| 3α-Hydroxy-5β-androstan-17-one | Bush et al. (1968) |

Another group of inhibitors for use according to the method of the invention is that described by the following formula: 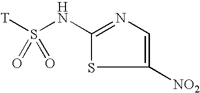
wherein T is an aryl ring, substituted with at least one of C1 6-alkyl, halogen, aryl or aryloxy, wherein the aryloxy residue can be further optionally substituted in one or more positions independently of each other by cyano and halogen; with the proviso that T is not 4-methylphenyl, 4-tert-butylphenyl, 4-chlorophenyl, and 4-fluorophenyl; as well as pharmaceutically acceptable salts, hydrates and solvates thereof.

Specific examples of those inhibitors described by the formula above are: 4 (3-chloro-2-cyanophenoxy)-N-(5-nitro-1,3-thiazol-2-yl)benzenesulfonamide; 3-chloro-2-methyl-N-(5-nitro-1,3-thiazol-2-yl)benzenesulfonamide; N-(5-nitro-1,3-thiazol-2-yl) [1,1′-biphenyl]-4-sulfonamide; N-(5-nitro-1,3-thiazol-2-yl)-4-n-propylbenzenesulfonamide; N-(5-nitro-1,3-thiazol-2-yl)-2,4,6-trichlorobenzenesulfonamide; 2,4-dichloro-6-methyl-N-(5-nitro-1,3-thiazol-2-yl)benzenesulfonamide.

The preparation and details of this group of inhibitors is provided in WO0190093 which is herein incorporated by reference.

A further group of 11HSD1 inhibitors suitable for use according to the invention are those described by the formula shown below and detailed in WO03/104207. 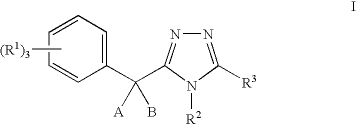
or a pharmaceutically acceptable salt or solvate thereof, wherein: A and B may be taken separately or together; when taken separately, A represents halo, C1-6alkyl, OC1-6alkyl or phenyl, said alkyl, phenyl and the alkyl portion of OC1 6alkyl being optionally substituted with 1-3 halo groups; and B represents H, halo, C1 6alkyl, —OC1 6alkyl, —SC1 6alkyl, C2-6alkenyl, phenyl or naphthyl, said alkyl, alkenyl, phenyl, naphthyl, and the alkyl portions of —OC1-6alkyl and —SC1-6alkyl being optionally substituted with 1-3 groups selected from halo, OH, CH30, CF3 and OCF3; and when taken together, A and B together represents (a) Cialkylene optionally substituted with 1-3 halo groups, and 1-2 Ra groups wherein Ra represents C1-3alkyl, OC1-3alkyl, C6-ioarC1-6alkylene or phenyl optionally substituted with 1-3 halo groups, or (b) Cz-5alkanediyl such that they form a 3-6 membered ring with the carbon atom to which they are attached, said ring optionally containing 1 double bond or 1-2 heteroatoms selected from 0, S and N, said 3-6 membered ring being optionally substituted with CI-4alkylene, oxo, ethylenedioxy or propylenedioxy, and being further optionally substituted with 14 groups selected from halo, C1-4alkyl, haloC1-4-alkyl, C1-3acyl, C1-3acyloxy, CI-3alkoxy, CI-6alkylOC(O)—, C2-4alkenyl, C2-4alkynyl, C1-3alkoxyC1-3alkyl, and R3 is selected from the group consisting of: C1 z4alkyl, C2-10alkenyl, SC1˜6alkyl, C6-ioaryl, heterocyclyl and heteroaryl, said alkyl, alkenyl, aryl, heterocyclyl, heteroaryl and the alkyl portion of S1-6alkyl being optionally substituted with (a) R; (b) 1-6 halo groups and (c) 1-3 groups selected from OH, NH2, NHCz 4alkyl, N (C1-4alkyl)2, C1-4alkyl, OC1-4alkyl, CN, C1 4alkylS (O) x-wherein x is 0, 1 or 2, C1-4-alkylSO2NH—, H2NS02-, C1-4alkylNHSO2- and (C1-4alkyl)2NSO2-, said Ci. 4alkyl and the A and B together represents (a) C1-4alkylene optionally substituted with 1-3 halo groups, and 1-2 Ra groups wherein Ra represents C1-3alkyl, OC1-3alkyl, C6-10arC1-6alkylene or phenyl optionally substituted with 1-3 halo groups, or (b) Cz-5alkanediyl such that a 3-6 membered ring is formed with the carbon atom to which they are attached, said ring being optionally interrupted with 1 double bond or 1-2 heteroatoms selected from 0, S, and N, said 3-6 membered ring being optionally substituted with C1-4alkylene, oxo, ethylenedioxy or propylenedioxy, and being further optionally substituted with 1-4 groups selected from halo, C1-4alkyl, haloCI-4alkyl, C1-3acyl, C1-3aryloxy, C-3alkoxy, C1-6alkylOC(O)—, C2-4alkenyl, C2-4alkynyl, C1-3alkoxyC1˜3alkoxyC1˜3allcoxy, phenyl, CN, OH, D, NH2, NHRa and N(Ra) 2 wherein Ra is as previously defined; each Ri represents H or is independently selected from the group consisting of: OH, halo, —C1-10alkyl, C1-6alkoxy and C6-10aryl, said C1-loalkyl, C6-ioaryl and the alkyl portion of C1 6alkoxy being optionally substituted with 1-3 halo, OH, OC1-3alkyl, phenyl or naphthyl groups, said phenyl and naphthyl being optionally substituted with 1-3 substituents independently selected from halo, OCH3, OCF3, CH3, CF3 and phenyl, wherein said phenyl is optionally substituted with 1-3 halo groups, or two R1 groups taken together represent a fused C5 6alkyl or aryl ring, which may be optionally substituted with 1-2 OH or Ra groups, wherein Ra is as defined above; R2 and R3 are taken together or separately; when taken together, R2 and R3 represent (a) a C3-8 alkanediyl forming a fused 5-10 membered non-aromatic ring optionally interrupted with 1-2-double bonds, and optionally interrupted by 1-2 heteroatoms selected; from 0, S and N; or (b) a fused 6-10 membered aromatic monocyclic or bicyclic group, said alkanediyl and aromatic monocyclic or bicyclic group being optionally substituted with 1-6 halo atoms, and 1-4 of OH, C1-3alkyl, OC1-3alkyl, haloC1-3alkyl, haloC1-3alkoxy, and phenyl, said phenyl being, optionally substituted with 1-4 groups independently selected from halo, C1-3alkyl, OC1-3alkyl, and said C1 3alkyl and the C1 3alkyl portion of OC1-3alkyl being optionally substituted with 1-3 halo groups; when taken separately,
R2 is selected from the group consisting of: (a) C1 14 alkyl optionally substituted with 1-6 halo groups and 1-3 substituents selected from OH, OC1 3alkyl, and phenyl, said phenyl being optionally substituted with 1-4 groups independently selected from halo, OCH3, OCF3, CH3 and CF3, and said C1 3 alkyl portion of OC 3alkyl being optionally substituted with 1-3 halo groups; (b) phenyl or pyridyl optionally substituted with 1-3 halo, OH or Ra groups, with Ra as previously defined; (c) C2 10 alkenyl, optionally substituted with 1-3 substituents independently selected from halo, OH and OC1 3alkyl, said C1 3 alkyl portion of OC1-3alkyl being optionally substituted with 1-3 halo groups; (d) CH2C02H; (e) CH2C02C1-6alkyl; (f) CH2C(O)NHRa wherein Ra is as previously defined; (g) NH2, NHRa and N(Ra) 2 wherein Ra is as previously defined, and R3 is selected from the group consisting of: C1 14alkyl, C2 10alkenyl, SC1 6alkyl, C6 loaryl, heterocyclyl and heteroaryl, said alkyl, alkenyl, aryl, heterocyclyl, heteroaryl and the alkyl portion of SC1-6alkyl being optionally substituted with (a) R; (b) 1-6 halo groups and (c) 1-3 groups selected from OH, NH2, NHC1-4alkyl, N(Ci. 4alkyl)2, C1-4alkyl, OC1-4alkyl, CN, C1-4alkylS (O) X wherein x is 0, 1 or 2, C1-4alkylSO2NH—, H2NSO2-, C1-4alkylNHSO2- and (C1x-4-alkyl)₂NSO2-, said C1 4alkyl and the C1-4alkyl portions of said groups being optionally substituted with phenyl and 1-3 halo groups, and R is selected from heterocyclyl, heteroaryl and aryl, said group being optionally substituted with 1-4 groups selected from halo, C1 4alkyl, C1 4alkylS (O) x-, with x as previously defined, C1˜4 alkyl S02NH—, H2NS02-, Ci-4alkylNHS02-, (C1-4 alkyl) 2NSO2-, CN, OH, OC1-4alkyl, and, said C1 4alkyl and the C1-4alkyl portions of said groups being optionally substituted with 1-5halo and 1 group selected from OH and OC1 3alkyl.

A further group of 11HSD1 inhibitors is provided by the group having the structural formula shown below and which are described in WO02072084 which is herein incorporated by reference. These inhibitors are described as GLYCYRRHETINIC ACID DERIVATIVES, PROGESTERONE AND PROGESTERONE DERIVATIVES 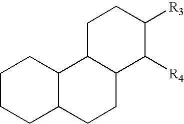
wherein R3 and R4 together define one or more rings, wherein the compound is substituted with one or more groups which are or which contain —OH or ═O.

A further group of 11HSD1 inhibitors are disclosed in WO0190091 which is herein incorporated by reference. Such inhibitors are described by the formula shown below: 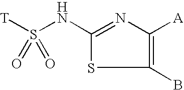
Wherein:
T is a monocyclic aryl ring or monocyclic heteroaryl ring, optionally independently substituted by [R] nn wherein n is an integer 0-5, and R is hydrogen, C1 6-alkyl, halogen, aryl or aryloxy, wherein the aryloxy residue can further be optionally substituted in one or more positions independently of each other by cyano and halogen; with the proviso: that when A is methyl and B is hydrogen, then T is not phenyl; that when A is methyl and B is 2,2,2-trichloroethyl, then T is not 4-methylphenyl; that when A is methyl and B is hydrogen, then T is not 4-bromophenyl; that when A is tert-butyl and B is bromo, then T is not 4-chlorophenyl; optionally also when A is methyl and B is hydrogen, then T is not 4-methylphenyl; optionally also when A is methyl and B is hydrogen; then T is not 4-chlorophenyl; and optionally also when A is methyl and R is 2,2,2-trichloroethyl, then T is not 4-methylphenyl.

A is C1 s-alkyl, vinyl or 3-(ethyl 3-methylbutanoate); B is hydrogen, methyl, ethyl, n-propyl, n-butyl, halogenatedC1 6-alkyl, C1 6-acyl or Ci-6-alkoxycarbonyl; as well as pharmaceutically acceptable salts, hydrates and solvates thereof.

A still further group of inhibitors are disclosed in WO190092 which is herein incorporated by reference. Also disclosed therein are details for their preparation and administration. Such inhibitors have the formula shown below: 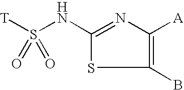
wherein
T is selected from thienyl substituted with one or more of bromo, chloro; phenyl substituted as follows: a) either T is phenyl, wherein the phenyl is substituted with one or more of propyl and phenyl; b) T is phenyl substituted with chloro in position 3 and methyl in position 2; c) T is phenyl substituted with chloro in position 2 and 4, and methyl in position 6; d) T is phenyl substituted with bromo in position 4 and fluoro in position 2 and 5; e) T is phenyl substituted with chloro in position 2, 3, and 4; f) T is phenyl substituted with chloro in position 2, 4, and 5; g) T is phenyl substituted with bromo in position 4 and methyl in position 2; h) T is phenyl substituted with chloro in position 2 and 6; i) T is phenyl substituted with chloro in position 2, 4, and 6; or j) T is phenyl substituted with bromo in position 4 and chloro in position 5.
A is selected from an aryl ring or heteroaryl ring, which can further be optionally substituted in one or more positions independently of each other by hydrogen, C1-6-alkyl, halogenatedC1 6-alkyl, halogen, C, 6-aLkoxy, nitro, C1 6-aLkoxycarbonyl, C aLkylsulfonyl, acetylamino or aryloxy, wherein the aryloxy can further be optionally substituted in one or more positions independently of each other by hydrogen and halogen;
B is selected from hydrogen and C1 6-aLkoxycarbonyl or is linked to A to give a 6membered aromatic or non-aromatic ring; as well as pharmaceutically acceptable salts, hydrates and solvates thereof.

Other suitable 11HSD1 inhibitors for use according to the present invention will be known to those skilled in the art. For the avoidance of doubt, suitable 11HSD1 inhibitors for use according to the methods of the invention are disclosed in the following patents and applications: WO 2004/033427, WO 2004/041264, WO 2004/011410 in the name of Astrazeneca; WO 2003/104207, WO 03/104208, WO 03/065983, WO2004058741 and US 2004/0048912 in the name of Merck WO 2004/037251, WO02072084 in the name of Sterix and WO0190090, WO0190091, WO0190092, WO0190093, WO0190094 in the name of Biovitrum and WO 2004/056745 in the name of Janssen. Those inhibitors disclosed in these applications and patents are suitable for use according to the present invention and those documents and inhibitors mentioned therein are herein incorporated by reference.

Administration of 11HSD1 Inhibitors and Compositions According to the Invention.

In a further aspect the present invention provides an agent selected from the group consisting of: thrombolytic agents; Glycoprotein IIb-IIIa platelet inhibitors, Calcium channel blockers, Antiarrythmics: Amiodarone and Lidocaine, Unfractionated heparin (UFH) or low-molecular-weight heparin (LMWH), Nitrates, Beta-blockers and Antiplatelets in conjunction with an 11HSD1 inhibitor and a pharmaceutically acceptable carrier, diluent or excipient.

According to the above aspect of the invention, preferably the 11HSD1 inhibitor is one of those disclosed herein.

The pharmaceutical compositions and inhibitors described herein may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by A mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

If the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example, intravenously, intramuscularly or subcutaneously. For parenteral administration, the compositions may be best used in the form of a sterile aqueous solution which may contain other substances, for example, enough salts or monosaccharides to make the solution isotonic with blood. For buccal or sublingual administration the compositions may be administered in the form of tablets or lozenges which can be formulated in a conventional manner.

The inhibitors and composition thereof disclosed herein may be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The pharmaceutical composition comprising the inhibitors and compositions thereof disclosed herein may also be used in combination with conventional treatments for the disease of interest.

Administration

The inhibitors and compositions thereof disclosed herein may be administered in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents—such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) may include, but are not limited to, one or more of oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Dose Levels

Typically, a physician will determine the actual dosage which will be most suitable for an individual subject.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Formulation

The non-viral delivery vectors may be formulated into a pharmaceutical composition, such as by mixing with one or more of a suitable carrier, diluent or excipient, by using techniques that are known in the art.

The invention will now be described by way of the following examples which should in no way be considered limiting of the invention.

Significance of the Invention Described Herein.

The present inventors have shown that the angiostatic effect of 11HSD1 inhibtors occurs at physiological concentrations of glucocorticoids and is mediated by glucocorticoid receptors, and that endogenous glucocorticoids tonically repress angiogenic responses. Glucocorticoid receptor blockade did not directly influence angiogenesis in isolated aortic rings because the inventors used serum-free media and hence steroids were absent. However, blockade of glucocorticoid receptors in vivo in mice increased angiogenesis in implanted subcutaneous sponges.

These observations raise the intriguing possibility that variations in cortisol levels, or in tissue sensitivity to cortisol, are a key determinant of the angiogenic component in diverse diseases. It is well recognised that Cushing's syndrome is associated with impaired wound healing. More recently, the inventors showed that exogenous glucocorticoid therapy is associated not only with increased incidence of myocardial infarction but also with an unexpected increase in prevalence of heart failure, suggesting an impact on the outcome as well as the incidence of cardiovascular disease. More subtle variations in cortisol secretion and action, including variations in responses to stress, have been described in many populations and related to risk factors for occlusive vascular disease, mood, development in early life, gender, age etc. The inventors now consider that the effects of cortisol on angiogenesis could explain the links between these quantitative traits in the population and the health outcomes from vascular disease, and perhaps from other diseases involving angiogenesis, including neoplasia. As such they consider that therapies which reduce glucocorticoid action within ischaemic tissue are valuable in improving collateral perfusion.

The present inventors described the presence of 11βHSD-1 in the vessel wall more than 10 years ago, but its importance has remained obscure. The observations that non-selective 11βHSD inhibitors influence vascular tone can be attributed to effects on the 11βHSD-2 isozyme which catalyses inactivation of glucocorticoids within endothelial cells. Here, the inventors show that regeneration of glucocorticoids by 11βHSD-1 in isolated aortae amplifies their angiostatic effect. The inventors found no evidence that 11βHSD-2 influences angiogenesis in vitro since the non-selective 11βHSD inhibitor carbenoxolone did not potentiate the angiostatic effect of corticosterone. In vivo 11βHSD-1 null mice have no obvious difference in vascular structure in healthy tissues. Normal vascular development occurs in other models of altered angiogenesis in which the abnormality is apparent only in adult pathology thus reflecting the distinct pathways underlying vasculogenesis and adult angiogenesis. However, when angiogenesis is stimulated in adult mice the inventors found that 11βHSD-1 amplifies the angiostatic effect of endogenous glucocorticoids. In subcutaneous sponge implants this is a local rather than systemic effect, since angiogenesis in contralateral sponges was unaffected. Moreover, cortisol concentrations in the sponges were lower following impregnation with cortisone than with cortisol, suggesting that it is generation of cortisol locally within the cells which express 11βHSD1, rather than levels of cortisol in the interstitial fluid of the sponge, which determines the angiostatic effect. Finally, the relevance of 11βHSD-1 was confirmed by the demonstration that 11βHSD-1 null mice exhibit greater angiogenic responses in wounds and in infarcted myocardium.

It is possible that these observations reflect 11βHSD-1 activity either within the vessel wall or in the inflammatory infiltrate which accompanies angiogenesis in all of these in vivo models. 11βHSD-1 is expressed in macrophages, and regeneration of glucocorticoids enhances phagocytosis of apoptotic neutrophils hence absence of 11βHSD-1 may confer a prolonged and enhanced acute inflammatory response which in turn might stimulate angiogenesis. However, 11βHSD-1 in the inflammatory infiltrate cannot explain the influence of 11βHSD-1 in isolated aortic rings. Nonetheless, inflammatory cytokines induce 11βHSD-1 expression in a variety of cell types including in vascular smooth muscle cells, so that the contribution of 11βHSD-1 within the vessel wall may be intimately related with the extent of the inflammatory response.

Angiogenesis is crucially dependent upon endothelial cells producing key factors such as vascular endothelial growth factor (VEGF) and forming a de novo collagen basement membrane to allow structured cell proliferation. In the chick chorioallantoic membrane, glucocorticoids alter endothelial cell morphology and collagen production It has also been proposed that glucocorticoid effects are mediated by inhibition of endothelial VEGF transcription and endothelial nitric oxide production. However, in keeping with a role for 11βHSD-1, the effect of glucocorticoids may be mediated within vascular smooth muscle, where inhibition of matrix metalloproteinase production may alter the efficacy of endothelium-dependent new vessel formation, and anti-proliferative effects may attenuate formation of vessel walls around endothelial cell buds.

In addition to enhanced angiogenesis in the infarcted myocardium, 11βHSD-1 null mice exhibited improved cardiac function after coronary artery ligation. Increasing the angiogenic response, and hence the collateral blood supply to the infarct, has been identified as a therapeutic objective in improving outcome from myocardial infarction However, this is not the only possible explanation for protective cardiac remodelling in 11βHSD-1 null mice. These mice have improved insulin sensitivity and lipid profile; these metabolic characteristics have been proposed to underlie the beneficial effects of insulin therapy on survival in diabetic patients after myocardial infarction 11βHSD-1 in liver and adipose tissue may also maintain angiotensinogen production and hence influence the degree of secondary hyperaldosteronism after infarction. Nevertheless, the current findings suggest that pharmacological inhibition of 11βHSD-1 may be valuable immediately after myocardial infarction in improving cardiac function. 11βHSD-1 inhibitors are already being developed for reducing risk factors for cardiovascular disease including in type 2 diabetes mellitus and obesity, and may find additional application in patients with established coronary artery disease.

This invention will now be described by way of the examples which should be considered in no way limiting of the invention.

### EXAMPLES

### Example 1

### Methods

Mice

Male, C57B16J wild type and 11HSD-1 homozygous null (−/−) mice aged 8-10 weeks were used (Charles River, UK). Genetic inactivation of 11βHSD-1 has been described in MF-1/129 mice¹²; for the current experiments mice were backcrossed over more than 10 generations onto a C57B16J background¹³.

Aortic Ring Preparations

Mice were killed humanely and thoracic aortae were removed, washed in serum free MCDB 131 medium (Invitrogen, UK), cleaned of periadventitial tissue and divided into 1-3 mm rings.

11βHSD activities were measured by incubating wild type aortic rings for 24 hours at 37° C. in 1 ml of DMEM-F12 medium (Invitrogen, UK) containing ³H-steroid supplemented with fetal bovine serum (1%), streptomycin (100 μg/ml), penicillin (100 units/ml) and amphotericin (0.25 μg/ml). 11 beta-Reductase activity was determined by adding 10 pmol [³H₄]-11-dehydrocorticosterone (synthesised in house from 1,2,6,7-[³H₄]-corticosterone (Amersham Biosciences, UK) using rat placental homogenate. Mouse liver (28±5 mg) and medium alone were used as positive and negative controls, respectively. 11beta-Dehydrogenase activity was determined by adding 10 pmol 1,2,6,7-[³H₄]-corticosterone. Mouse kidney (13±3 mg) and medium alone served as positive and negative controls. After incubation, steroids were extracted from media using C₁₈ Sep-pak columns (Waters Millipore, UK). Aortic rings, which contain only 2-3% of the added radioactivity, were not included in the extraction. [³H₄]-Corticosterone and [³H₄]-11-dehydrocorticosterone were separated by HPLC and quantified by on-line liquid scintillation counting. Enzyme activity was expressed as conversion after subtraction of apparent conversion in negative control wells.

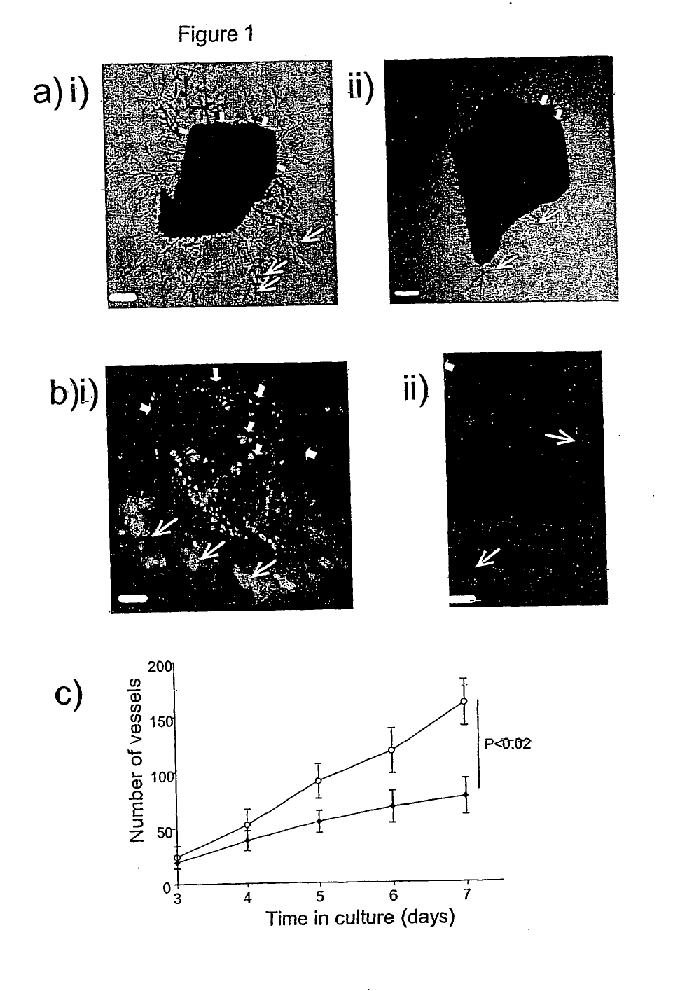

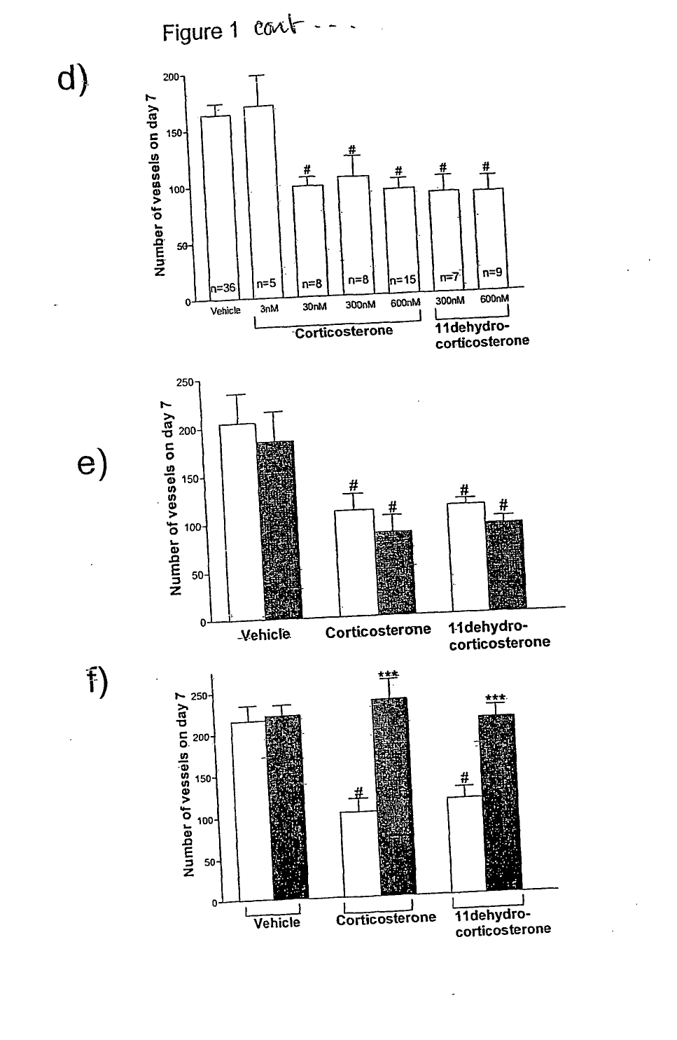

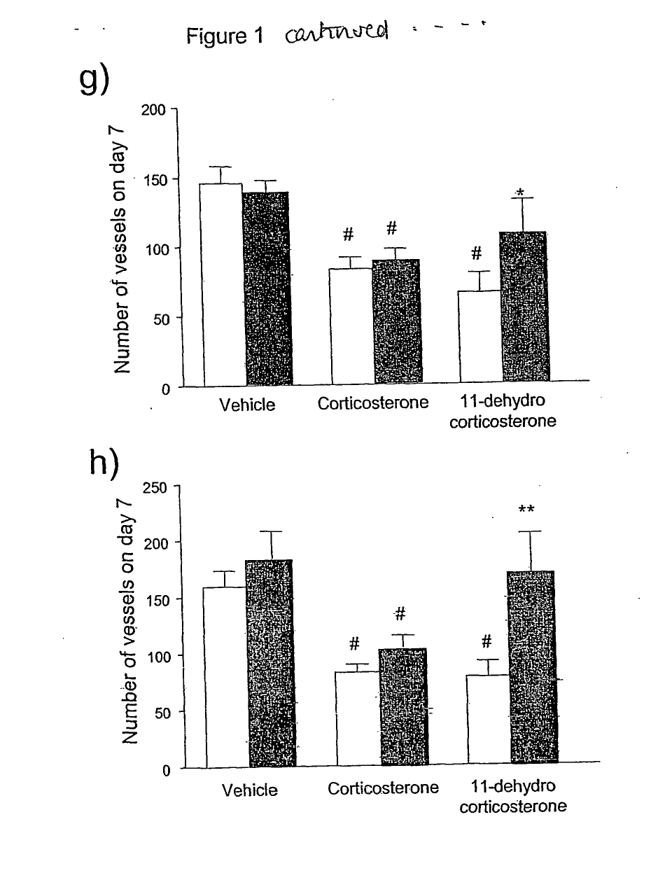

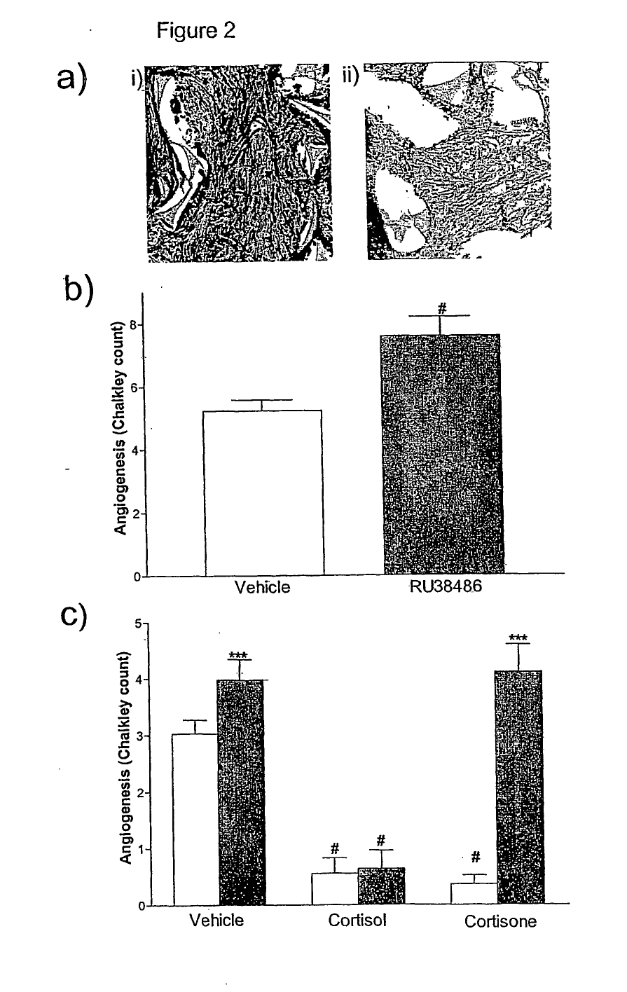

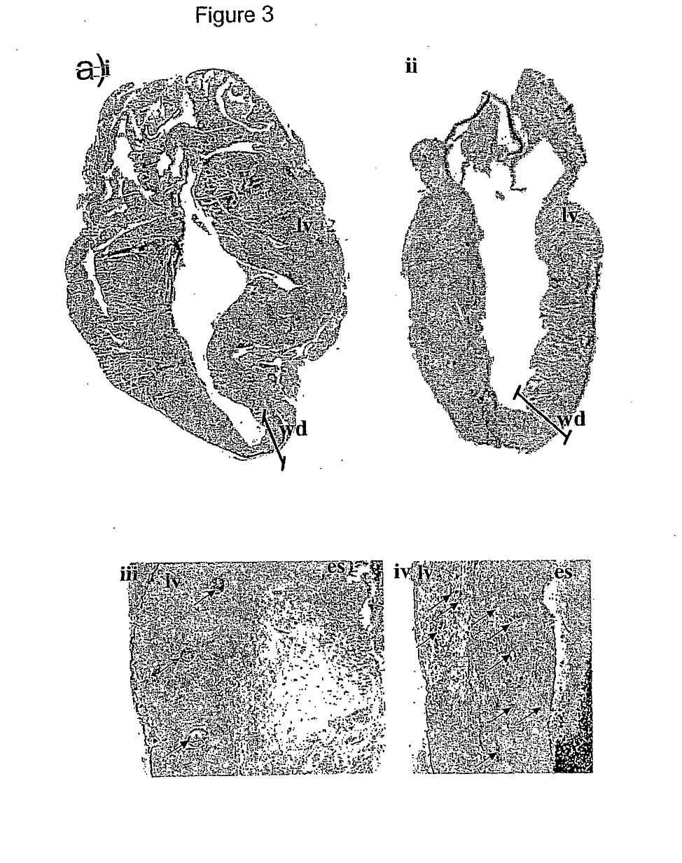

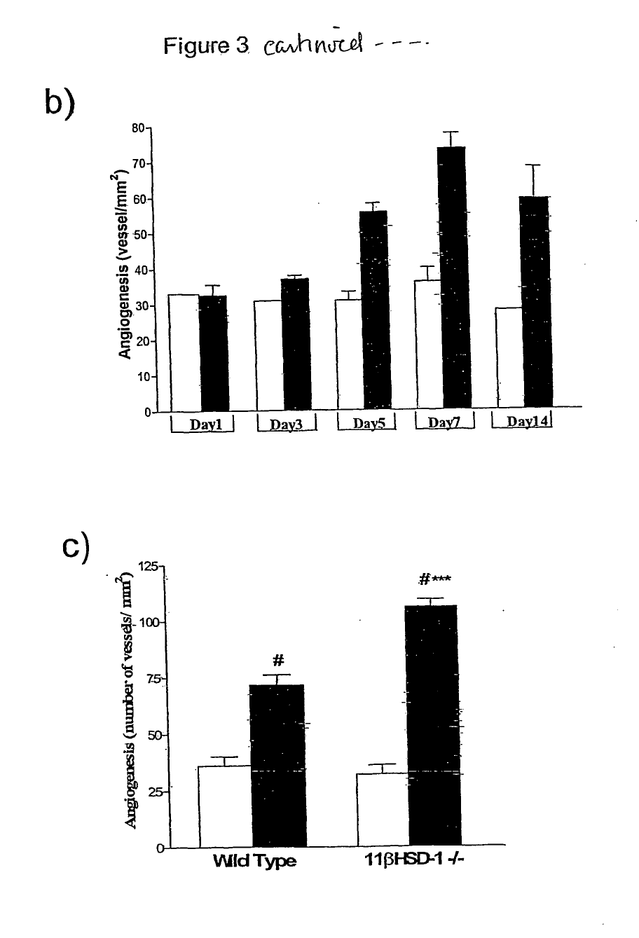

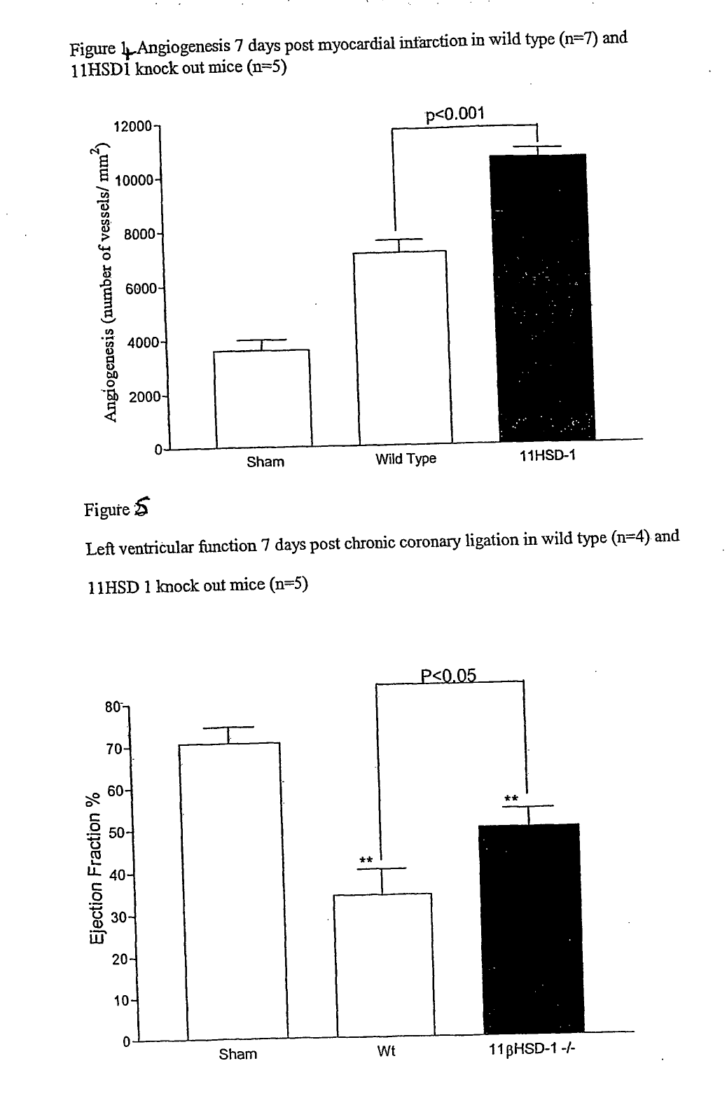

To quantify angiogenesis aortic rings were embedded in 200 μl of steroid free Matrigel (Becton Dickinson, UK) and incubated at 37° C. in serum free MCDB 131, with heparin, ascorbic acid, and GA1000 (Cambrex Biosciences, UK) in the presence and absence of: corticosterone (3 nM, 30 nM, 300 nM, 600 nM); 11-dehydrocorticosterone (300 nM, 60 nM); the glucocorticoid receptor antagonist, RU38486 (10⁻⁶M)⁵⁹, the mineralocorticoid receptor antagonist, spironolactone (10⁻⁶ M)⁶⁰; and/or the non-selective selective 11βHSD inhibitor, carbenoxolone (10⁻⁴-10⁻⁶M)³¹. All drugs (Sigma-Aldrich, UK) were dissolved in ethanol and diluted in aqueous solution; final ethanol concentration 1-3% v/v. Media were changed every 48 hours. Experiments were performed in triplicate. In initial experiments, new vessels were counted daily using light microscopy (FIG. 1). From these studies day 7 was selected as the appropriate time point to examine the effects of glucocorticoids (FIG. 1c).

To confirm the nature of apparent new vessels, endothelial cells were identified by uptake of fluorescent-labelled acetylated low-density lipoprotein (DiI-Ac-LDL)(Biogenesis, Dorset UK) FIG. 1b).

Subcutaneous Sponge Implant Assay

Mice were anaesthetized with halothane and a sterilised sponge cylinder (0.5 cm×1 cm) (Caligen-Foam Ltd., UK) was implanted subcutaneously on each flank. Sponges contained a silastic insert (Silastic 20 medical grade, Dow Corning Corporation, USA) impregnated with vehicle, 2 mg of cortisol or cortisone, or 5 mg of RU38486. Each animal had an intervention-impregnated sponge (steroid or RU38486) on one side and a placebo-impregnated sponge (silastic only) on the other. Such inserts release their impregnated compounds in vivo at a constant rate for 3 weeks. Human steroids (cortisol and cortisone, equivalent to corticosterone and 11-dehydrocorticosterone) were used to allow distinction from endogenous steroids. In separate experiments (not shown) angiogenesis in placebo-impregnated sponges was not altered by the presence or absence of a contralateral steroid-treated sponge.

Twenty days following implantation, mice were decapitated, sponges excised and inserts removed. Sponges were bisected; one half was fixed in 10% formalin and embedded in paraffin wax. Sections (8 μm) were stained with haematoxylin and eosin (H&E). The second half of the sponge was weighed, homogenised in 2 ml sterile phosphate buffered saline at 4° C. and centrifuged (2000 g; 30 minutes). Steroids were extracted from the supernatant using ethyl acetate and cortisol quantified using a specific radioimmunoassay (Amersham Pharmacia Biotech, UK). Sponge vessel density was determined by using the mean of triplicate Chalkley counts on 2 sections per sponge.

Chronic Coronary Artery Ligation

Mice were anaesthetised with an intraperitoneal injection of xylazine (0.018 mg/kg), ketamine (100 mg/kg), and atropine (600 mcg/kg). Surgery was performed as previously described. Briefly; following endotracheal intubation and mechanical ventilation (Mini-Vent, Harvard Instruments, US) superficial tissues were dissected, an incision made in the 4^{th} intercostal space, the pericardium divided and the left main descending artery ligated with 6.0 prolene suture (Ethicon, UK). In sham operated animals the suture was not ligated. The thoracic wall was closed by layered suturing; the skin was stitched with a continuous suture using 5-0 Mersilk with a 10 mm 3/8c round-bodied needle (Ethicon, UK). On completion of surgery animals received intraperitoneal atipamazole (5 mg/kg) and subcutaneous buprenorphine (0.05 mg/kg).

Mice were sacrificed on days 1, 3, 5, 7 and 14 after surgery by cervical dislocation. Before sacrifice animals were re-anaesthetised and echocardiography was performed using a Diasus ultrasound machine (Dynamic Imaging, Livingston, UK). A 10-20 MHz transducer was applied parasternally to the shaved chest wall and maneuvered to obtain 2-dimensional (2-D) images in a parasternal long-axis view. Images were stored on an optical disk and analysed offline using Diasus software (Dynamic Imaging). Left ventricular parameters were measured on live 2-D images and averaged from 2 cardiac cycles. Excised hearts and wounds were fixed in 10% formalin, paraffin embedded and sectioned at 8 μm. Sections were stained with haematoxylin and eosin to measure infarct size, or anti-von Willebrand factor (Dakocytomation, UK) to label endothelial cells and quantify angiogenesis.

Ventricular Angiogenesis

Vessels were counted at ×400 magnification (Karl Ziess Axioskop) in the 4 most vascular fields (2 endocardial and 2 epicardial) using a 0.0625 mm² reticule; the borders of the reticule were within the infarct. Infarct size was measured as a percentage of left ventricular wall area. Measurements of infarct size were taken at direct light microscopy; images were captured using Research Systems Photometric camera and analysed using in-house scripts.

Cutaneous Angiogenesis

Wound vessel density was determined in the dermis at ×250 light microscopy using the mean of triplicate Chalkley counts on 2 sections per wound.

Statistics

Data are mean ±SEM. Comparisons were made by ANOVA with least squares difference post hoc tests. Inter-assay- and intra-assay coefficients of variation in wild type mice were 17% (n=32) and 22% (n=18), respectively for vessel number in aortic rings after 7 days in culture; 12% (n=6) and 12% (n=6) for vessel density in sponge implants; 7% (n=6) and 25% (n=6) in day 7 infarcts and 7% (n=4) and 12% (n=4) for day 7 wounds.

### Example 2

### In Aortic Rings In Vitro the Angiostatic Effect of Glucocorticoids is Mediated by Glucocorticoid Receptors and Amplified by 11βHSD-1

To test the direct effects of glucocorticoids on new vessel formation, independently of any in vivo inflammation, we quantified new vessel formation in mouse aortic rings embedded in Matrigel^{8,30}. The endothelial cell content of the new vessels was confirmed by uptake of fluorescent LDL and 7 days was chosen as an appropriate incubation time to detect effects of steroids (FIG. 1). Both corticosterone and 11-dehydrocorticosterone inhibited angiogenesis in wild type vessels across a range of physiological concentrations (FIGS. 1c and 1d). The mineralocorticoid receptor antagonist spironolactone had no effect on angiogenesis or on the angiostatic effects of corticosterone or 11-dehydrocorticosterone (FIG. 1e). The glucocorticoid receptor antagonist RU38486 did not affect angiogenesis directly, but prevented the angiostatic effects of both corticosterone and 11-dehydrocorticosterone (FIG. 1f).

To confirm activity of 11βHSD-1 in the aortic ring preparation, rings were incubated with ³H₄-corticosterone or ³H₄-11-dehydrocorticosterone and interconversion quantified by HPLC with on-line scintillation counting. Measurement of relevant product generation confirmed both 11β-reductase (0.65±0.24 pmol/mg) and 11μ-dehydrogenase (0.66±0.28 pmol/mg) activities in aortic rings with similar conversion rates as in positive controls; liver for 11βHSD-1 (0.18±0.03 pmol/mg) and kidney for 11βHSD-2 (2.13±1.65 pmol/mg). Pharmacological inhibition of 11βHSDs in aortic rings was achieved with the non-selective inhibitor carbenoxolone³¹. At high concentration (10⁻⁴M), carbenoxolone abolished angiogenesis, consistent with previously reported detrimental effects on endothelial cell growth³². At lower concentration (10⁻⁶M) carbenoxolone had no direct effect, and did not influence the angiostatic effect of corticosterone, but prevented the angiostatic effect of 11-dehydrocorticosterone (FIG. 1g).

Aortic rings were obtained from homozygous 11βHSD-1 null (−/−) mice on a C57B16 genetic background³³ and wild type controls. Angiogenesis in aortic rings from 11βHSD-1 −/− mice was similar to wild type controls in the absence of steroid and inhibited to a similar degree by corticosterone. However, in contrast with its angiostatic effect in wild type controls, 11-dehydrocorticosterone did not inhibit angiogenesis in vessels from 11βHSD-1 −/− mice (FIG. 1h).

### Example 3

### It Subcutaneous Sponge Implants In Vivo the Angiostatic Effect of Endogenous Glucocorticoids is Mediated by Glucocorticoid Receptors and Amplified by 11βHSD-1

To assess angiogenesis in vivo we implanted foam sponges subcutaneously¹² in both flanks of mice. The sponges contained silastic implants impregnated with steroid or drugs to allow local manipulation of glucocorticoid action; systemic effects were tested by comparison with contralateral sponges containing placebo silastic implants.

Placebo impregnated sponges excised after 20 days¹² were red on gross inspection with a lace-like covering of blood vessels. At histology there was an inflammatory infiltrate and an abundance of blood vessels (FIGS. 2a,i). In sponges impregnated with the glucocorticoid receptor antagonist RU38486 and implanted in C57B16 mice, the number of vessels in the sponge was increased (FIG. 2b), indicating a tonic angiostatic effect of endogenous glucocorticoids.

To test the effects of 11-hydroxy and 11-keto-glucocorticoids we used the ‘human’ steroids, cortisol and cortisone, which allowed measurement of steroid concentrations within the sponge independently of endogenous corticosterone and 11-dehydrocorticosterone (Table 1). Glucocorticoid-treated sponges from wild type C57B16 were white on gross inspection. At histology, there was an inflammatory infiltrate but few discernable vascular structures could be seen (FIGS. 2a,ii) and quantification confirmed that, analogous to the findings in aortic rings in vitro, both cortisol and cortisone inhibited angiogenesis in vivo (FIG. 2c). In 11βHSD-1 null mice angiogenesis was increased in placebo sponges, consistent with tonic amplification of the angiostatic effect of endogenous glucocorticoids by 11HSD-β1 in wild type animals. Impregnation with cortisol produced similar cortisol concentrations in wild type and 11βHSD-1 null mice (Table 1) and inhibited angiogenesis to a similar degree (FIG. 2c). However, impregnation with cortisone did not elevate sponge cortisol concentrations in 11βHSD-1 null mice, as it did in wild type controls (Table 1), and did not inhibit angiogenesis (FIG. 2c).

### Example 4

### 11βHSD-1 Null Mice have Enhanced Angiogenesis and Improved Left Ventricular Function Followed Myocardial Infarction

To establish the relevance of our observations in pathology, we examined angiogenesis in the myocardium of mice following ligation of the left main descending coronary artery. The angiogenic response was well established 7 days after infarction (FIG. 3b) so this interval was selected for comparisons. Echocardiography was performed before animals were sacrificed.

In 11βHSD-1 null mice 7 days after coronary artery ligation, the angiogenic response was greater (FIG. 3c). At echocardiography (Table 2) 11βHSD-1 null mice had less pronounced infarction-associated increase in left ventricular internal diameter, thinning of the left ventricular wall, and decrease in left ventricular ejection fraction. For example, left ventricular ejection fraction fell from ˜66% in sham operated animals to ˜20% in wild type mice but only to ˜35% in 11βHSD-1 null mice. Post mortem measurement of infarct size showed no difference in the efficacy of surgery in the two strains (44.2±3.4% for wild type (n=5) and 44.2±2.6% for 11βHSD-1 −/− mice (n=5).

### Example 5

### 11βHSD-1 Mice have Enhanced-Angiogenesis in Surgical Wounds

In mice that underwent thoracotomy as part of the coronary artery ligation study, had their cutaneous surgical wounds collected to examine new vessel formation in the healing skin. The dermal angiogenic response was greater in 11βHSD-1 null mice (5.1±0.27 Chalkey count), than in wild type mice (3.5±0.25 Chalkley count; p<0.01).

### Example 6

### Enhanced Myocardial Angiogenesis and Improved LVEF Following Coronary Artery Ligation in Mice Homozygous for a Null 11HSD1 Allele (Knockout Mice) Compared with Wild Type Controls

Studies were performed on 8-10 week old male C57B16 wild type mice and age and sex matched mice on the same genetic background which were homozygous for a disrupted 11HSD1 allele (knockout mice).

Surgery was performed under anaesthesia with ketamine, xylazine and atropine. The left main descending coronary artery was ligated or sham surgery was performed. Animals were allowed to recover and underwent echocardiography to measure left ventricular ejection fraction at 7 days after surgery. They were then killed and hearts obtained.

Echocardiograms were performed on a Diasus ultrasound machine (Dynamic Imaging Livingston UK) Images were analyzed offline using Diasus software Dynamic Imaging).

Hearts were fixed in formalin, paraffin embedded and sectioned, and stained with Haematoxylin & Eosin and using immunohistochemistry with an antibody against von Willebrand factor (to identify vessels easily). Vessels were counted in an area of 0.0625 mm² selected as previously described within the most vascular region of infarcted tissue ((10)).

FIG. 4 shows the numbers of myocardial vessels identified in the two groups following sham surgery and following coronary artery ligation. FIG. 4. Angiogenesis 7 days post myocardial infarction in wild type (n=7) and 11HSD1 knock out mice (n=5)

FIG. 5 shows the left ventricular ejection fraction in the two groups following sham surgery and following coronary artery ligation. * indicates significant difference versus sham surgery. Left ventricular function 7 days post chronic coronary ligation in wild type (n=5) and

11HSD 1 knock out mice (n=5).

TABLE 1

| | | Cortisol level (ng/g sponge) | |
|---|---|---|---|
| | Steroid | Ipsilateral steroid | Contralateral Placebo |
| Strain | Impregnated | treated sponge | treated sponge |
| Wild type | Cortisol | 4271 ± 186 # | 161 ± 18 |
| | Cortisone | 295 ± 25 #** | 98 ± 19 |
| 11bHSD-1 −/− | Cortisol | 3775 ± 1703 # | 135 ± 46 |
| | Cortisone | 87 ± 11 | 90 ± 30 |

TABLE 2

| | Wild type | | 11bHSD-1 −/− | |
|---|---|---|---|---|
| | Sham | Infarct | Sham | Infarct |
| | (n = 3) | (n = 5) | (n = 3) | (n = 5) |
| LVEDD(mm) | 3.3 ± 0.1# | 4.7 ± 0.1 | 3.0 ± 0.2# | 4.2 ± 0.3 |
| LVESD(mm) | 1.9 ± 0.1# | 4.1 ± 0.2 | 1.7 ± 0.2# | 3.2 ± 0.4* |
| LVareaED | 15.4 ± 1.8# | 26.8 ± 1.5 | 13.2 ± 1.3 | 17.3 ± 1.4** |
| LVareaES | 5.1 ± 0.4# | 21.5 ± 1.1 | 4.5 ± 0.5# | 11.5 ± 1.2** |
| PWD(mm) | 1.1 ± 0.1# | 0.6 ± 0.1 | 1.3 ± 0.2 | 1.0 ± 0.2* |
| PWS(mm) | 1.4 ± 0.1# | 0.8 ± 0.1 | 1.6 ± 0.2 | 1.4 ± 0.1* |
| FS | 42.6 ± 2.8# | 13.4 ± 1.0 | 43.1 ± 0.5# | 25.3 ± 3.3** |
| EF % | 66.0 ± 5.5# | 19.5 ± 1.4 | 64.3 ± 5.6# | 35.2 ± 4.7* |

This example shows that the inhibition of 11HSD1 during and for at least 1 week after myocardial inaction enhances angiogenesis and hence collateral blood supply to ischaemic and infarcted areas in the myocardium following coronary artery occlusion. This is associated with favourable left ventricular remodelling with enhanced LVEF, which predicts improved morbidity and mortality following myocardial infarction.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, molecular biology and biotechnology or related fields are intended to be within the scope of the following claims.

### REFERENCE LIST

(1) Owen A R, Stables R H. Myocardial revascularisation by laser. Int J Cardiol 2000; 72(3):215-220.
(2) Kleiman N S, Patel N C, Allen K B, Simons M, Yla-Herttuala S, Griffin E et al. Evolving revascularization approaches for myocardial ischemia. Am J Cardiol 2003; 92(9B):9N-17N.
(3) Hasan Q, Tan S T, Gush J, Peters S G, Davis P F. Steroid therapy of a proliferating hemangioma: histochemical and molecular changes. Pediatrics 2000; 105(1 Pt 1):117-120.
(4) Versaci F, Gaspardone A, Tomai F, Ribichini F, Russo P, Proietti I et al. Immunosuppressive Therapy for the Prevention of Restenosis after Coronary Artery Stent Implantation (IMPRESS Study). J Am Coll Cardiol 2002; 40(11):1935-1942.
(5) Walker B R, Yau J L, Brett L P, Secki J R, Monder C, Williams B C et al. 11β-Hydroxysteroid dehydrogenase in vascular smooth muscle and heart: implications for cardiovascular responses to glucocorticoids. Endocrinology 1991; 129:3305-3312.
(6) Young M, Funder J. Mineralocorticoid action and sodium-hydrogen exchange: studies in experimental cardiac fibrosis. Endocrinology 2003; 144(9):3848-3851.
(7) Hadoke P W F, Christy C, Kotelevtsev Y V, Williams B C, Kenyon C J, Seckl J R et al. Endothelial cell dysfunction in mice after transgenic knockout of type 2, but not type 1, 11β-hydroxysteroid dehydrogenase. Circulation 2001; 104(23):2832-2837.
(8) Christy C, Hadoke P W F, Paterson J M, Mullins J J, Seckl J R, Walker B R. Glucocorticoid action in mouse aorta; localisation of 11beta Hydroxysteroid dehydrogenase type 2 and effects on response to glucocorticoid in vitro. Hypertension 2003; 42(4):580-587.
(9) Cai T Q, Wong B M, Mundt S S, Thieringer R, Wright S D, Hermanowski-Vosatka A. Induction of 11beta-hydroxysteroid dehydrogenase type 1 but not type 2 in human aortic smooth muscle cells by inflammatory stimuli. J Steroid Biochem 2001; 77(2-3):117-122.
(10) Virag J I, Murry C E. Myofibroblast and endothelial cell proliferation during murine myocardial infarct repair. Am J Pathol 2003; 163(6):2433-2440.
(11) Small, G. R., Hadoke, P. W. F. & Walker, B. R. Glucocorticoids inhibit angiogenesis in mouse aorta: a novel role for 11βHSD1 in modifying vascular structure. Presented at the American Endocrine Society 85th Annual Meeting. San Francisco, June 2003.
(12) Kotelevtsev, Y. V. et al. 11β-Hydroxysteroid dehydrogenase type 1 knockout mice show attenuated glucocorticoid inducible responses and resist hyperglycaemia on obesity and stress. Proc. Natnl. Acad. Sci. USA 94, 14924-14929 (1997).
(13) Morton, N. M. et al. Novel adipose tissue-mediated resistance to diet-induced visceral obesity in 11 beta-hydroxysteroid dehydrogenase-type 1-deficient mice. Diabetes 53, 931-938 (2004).

1. A method for the treatment of myocardial infarction in patient which method comprises administering to a patient in need of such treatment an inhibitor of 11HSD1.

2. A method for the treatment of mycardial infarction in a patient comprising administering an inhibitor of 11HSD1.

3. A method for increasing the left ventricular ejection fraction following myocardial infarction comprising administering an inhibitor of 11HSD1.

4. A method of enhancing myocardial remodeling post myocardial infraction in a patient comprising administering an inhibitor of 11HSD1.

5. A method of enhancing myocardial angiogenesis following myocardial infarction in a patent comprising administering an inhibitor of 11HSD1.

6. The method according to any of claims 1 to 5 wherein the inhibitor is carbenoxoline.

7. A composition comprising, preferably consisting of, an inhibitor of 11HSD1 and one or more agents used in the routine treatment of myocardial infarction and a pharmaceutically acceptable carrier, diluent and/or exipient.

8. A composition according to claims 7 wherein the one or more agents used routinely in the treatment of myocardial infarction is one or more of those agents in the group consisting of: thrombolytic agents; Glycoprotein Iib0IIIa platelet inhibitors, calcium channel blockers, Antiarrythmics: Amiodarone and Lidocaine, Unfractionated heparin (UFH) or low-molecular-weight heparin (LMWH), Nitrates, Beta-blockers, Angiotensin receptor blockers, and angiotensin converting enzyme inhibitors.

9. A compositions according to claim 7 or claim 8 wherein the 11HSD1 inhibitor is carboxenolone.

10. A method for the treatment of myocardial infarction in a patient which method comprises the step of administering to a patient in need of such treatment, either sequentially or simultaneously an inhibitor of 11HSD1 in conjunction with one or more agents selected form the group consisting of the following: thrombolytic agents; Glycoprotein IIb-IIIa platelet inhibitors, Calcium channel blockers, Antiarrythmics: Amiodarone and Lidocaine, Unfractionated heparin (UFH) or low-molecular-weight heparin (LMWH), Nitrates, Beta-blockers, angiotensin receptor blockers, and angiotensin converting enzyme inhibitors.

11. A method according to claim 10 wherein the one or more agents is thrombolytic agent.

12. A method according to claim 11 wherein the thrombolytic agent is TPA or streptokinase.
